(19) United States
(12) Patent Application Publication
(10) Pub. No.: US 2022/0249102 A1
(43) Pub. Date: Aug. 11, 2022

(54) VARIABLE DIAMETER REAMER

(71) Applicant: MEDTRONIC, INC., Minneapolis, MN (US)

(72) Inventors: Suresh Kumar Prema Mohanasundaram, Hyderabad (IN); Amit Sharad Bharos, Hyderabad (IN)

(73) Assignee: MEDTRONIC, INC., Minneapolis, MN (US)

(21) Appl. No.: 17/160,616

(22) Filed: Feb. 10, 2021

(51) Int. Cl. A61B 17/16

(52) U.S. Cl. CPC A61B 17/1617; A61B 17/320725; A61B 17/1666

(57) ABSTRACT

Various embodiments of a variable diameter calcar planer for reaming a boney surface and associated methods of using the same are disclosed. An example device may include a cylindrical body defining a longitudinal axis extending from a proximal end to a distal end and a positioning ring disposed at the distal end. Various devices may include plurality of primary blades that are radially disposed on the positioning ring that are configured for reaming at the first diameter. Various devices may include a collar disposed around the cylindrical body and axially aligned with the longitudinal axis, for example. Some devices may further include a plurality of secondary blades, configured to expand a reaming area by moving the collar towards the distal end. In various embodiments, the collar facilitates reaming of a boney surface at various diameters by moving the blades laterally outward with respect to the longitudinal axis.

### CROSS-REFERENCE TO RELATED APPLICATIONS

This application incorporates the disclosure of U.S. Pat. No. 10,456,122, titled Surgical system including powered rotary-type handpiece, filed Mar. 13, 2013 and granted Oct. 29, 2019.

### BACKGROUND

This disclosure relates to the field of Orthopedics, in particular, for example, to the sub-field of “Total Hip Arthroplasty” and the preparation of a calcar plane to facilitate the proper seating of a femoral stem at proximal femur. Relevant patient groups may include those having painful, disabling joint disease of the hip resulting from degenerative arthritis, rheumatoid arthritis, and/or post-traumatic arthritis. Another relevant patient group may include those having low or poor quality bone stock, and/or post trauma fractures. These patient groups may require and/or benefit from a Total Hip Arthroplasty (THA) procedure.

During THA, the damaged portions of a patient's hip joint may be removed. For example, the ball shaped femoral head may be removed and replaced with a prosthetic ball made of metal and/or ceramic, for example. The corresponding socket shaped acetabulum may be removed and replaced with a prosthetic cup, for example. The cup may consist of one or two components made of metal, ceramic, and/or plastic. A stem may also be placed in the femur to support the femoral head, for example. In turn, the femoral head may attach to the taper portion of the stem. As explained, these components are intended to restore mobility and/or relieve pain associated with any of the above mentioned conditions, for example.

Various methods may be performed by a surgeon in completing a THA. At least one method may include preparing a proximal femur and acetabulum in order to receive an implant, for example as described above. To prepare the femoral neck, a neck resection may be performed. The patient specific level of the femoral neck may be dependent on the pre-operative planning regarding the stem size selection and leg length adjustment, and the amount of bone stock that is available. By using the anatomic landmarks referenced during pre-operative x-ray templates, a pre-planned neck resection may be made with the aid of a Neck Resection Guide, for example. The Neck Resection Guide may assist a surgeon with determining the correct stem orientation and placement, for example. After careful pre-operative templating, the Neck Resection Guide may be placed on the anterior/posterior aspect of an exposed proximal femur and the planned femoral neck cut may be marked using a marking instrument.

Next, based on this marking, a resection may be performed with a cutting saw or bone saw, for example. After the resection is performed the femoral canal may be prepared. Preparation of the femoral canal may include drilling and reaming the bone surface through the femoral canal. This may create a cavity for the femoral stem, for example. Additionally, broaching may be performed through a series of broaches until a desired stem size is achieved. Due to the fact that the femoral neck is resected roughly by a cutting saw or bone saw, surface reaming is often required to ensure an adequate and smooth surface, for example. In the industry, a “calcar planer” may be used for this purpose. Due to significant differences in patient anatomy and various surgical designs, calcar plane reaming may need to need to be performed for various different diametrical ranges. For example, due to the patient's femur anatomy (or cross sectional area at the calcar region after neck resection).

After the calcar region has been reamed, a femoral stem may be inserted into the prepared cavity. Next, a femoral head may be assembled over the femoral stem, for example. Acetabular region may also be prepared for receiving the acetabular shell and liner, for example. The femoral stem assembly may then articulate inside the acetabular liner across the intended Range of Motion (ROM).

Conventional reaming tools can only ream a surface with a single fixed size or diameter. This requires a surgeon to have multiple reaming tools of various diameters and increases the total time required to perform a surgery and may increase the likelihood for introducing germs into a patient.

This disclosure describes improvements over these prior surgical methods and technologies. For example, an improved means to perform calcar reaming for a THA procedure via a variable diameter calcar reamer is disclosed. However, it shall be understand that the disclosure herein is not limited to just reaming of the calcar plane and can of course be applied to other similar situations with respect to different patient anatomy, for example.

### SUMMARY

The present disclosure relates to surgical instruments, surgical systems, and associated methods for operating the surgical instrument and the surgical systems.

In one aspect, the disclosure provides a variable diameter reaming device, for example. The reaming device may include a cylindrical body defining a longitudinal axis extending from a proximal end to a distal end, for example. The reaming device may further include a positioning ring disposed at the distal end of the cylindrical body that has a centrally disposed guide aperture, for example. The reaming device may further include a collar disposed around the cylindrical body and axially aligned with the longitudinal axis, and a plurality of radially disposed blades. In various embodiments, each blade may be supported by a corresponding platform, and each platform may be operably connected to the positioning ring by a sliding arm and may be operably connected to the collar by a link, for example. In some embodiments, the collar may be configured to move up and down the cylindrical body along the longitudinal axis thereby moving the plurality of blades between a retracted position and an extended position, for example. In some embodiments, in the retracted position, the plurality of blades are configured for reaming at a first diameter, and, in the extended position, the plurality of blades are configured for reaming at a second diameter that may be larger than the first diameter.

In another aspect, the disclosure provides that the cylindrical body further includes a stop ring disposed adjacent to the positioning ring that may be configured to stop the cylindrical collar in the extended position, for example.

In another aspect, the disclosure provides that the cylindrical body further includes a plurality of collar arms, and each collar arm may be coupled to a corresponding link by a pinned connection, for example.

In another aspect, the disclosure provides that each platform may be operably coupled to a corresponding link by a pinned connection, for example.

In another aspect, the disclosure provides that in some embodiments, each sliding arm may be configured to support and move each platform towards and away from the positioning ring in a lateral direction, for example. Additionally, the lateral direction may be substantially perpendicular with the longitudinal direction, for example.

In another aspect, the disclosure provides that the guide aperture may be configured to surround a broach stem thereby positioning the plurality of blades radially around the broach stem, for example.

In another aspect, the disclosure provides for a variable diameter reaming device. The reaming device may include a cylindrical body defining a longitudinal axis extending from a proximal end to a distal end, for example. The reaming device may further include a positioning ring disposed at the distal end of the cylindrical body that defines a first diameter, for example. The reaming device may further include a plurality of primary blades that are radially disposed on the positioning ring, the plurality of primary blades may be configured for reaming at the first diameter, for example. The reaming device may further include a collar disposed around the cylindrical body and axially aligned with the longitudinal axis, for example. The reaming device may further include a plurality of secondary blades, and each secondary blade may be supported by the positioning ring and a corresponding link. Additionally, each link may be operably connected to the collar, for example. In various embodiments, the collar may be configured to move up and down the cylindrical body along the longitudinal axis thereby moving the plurality of secondary blades between a retracted position and an extended position, for example. In various embodiments, in the retracted position, the plurality of primary blades may be configured for reaming at the first diameter and the plurality of secondary blades are retracted, for example. In various embodiments, in the extended position, the plurality of primary blades are configured for reaming at the first diameter and the plurality of secondary blades are configured for reaming at a second diameter, the second diameter may be larger than the first diameter, for example.

In another aspect, the disclosure provides that the positioning ring may include a centrally disposed guide rail and an outer rail, for example. The centrally disposed guide rail may define a centrally disposed aperture configured to surround a broach stem thereby positioning the plurality of primary blades and the plurality of secondary blades radially around the broach stem, for example.

In another aspect, the disclosure provides that each primary blade of the plurality of primary blades may extend from the centrally disposed guide rail to the outer rail, for example.

In another aspect, the disclosure provides that a cutting edge of each primary blade of the plurality of primary blades extends in a direction that may be substantially perpendicular to the longitudinal axis, for example.

In another aspect, the disclosure provides that in the extended position, a cutting edge of each secondary blade of the plurality of secondary blades extends in a direction substantially perpendicular to the longitudinal axis, for example.

In another aspect, the disclosure provides that in the extended position each cutting edge of each primary blade of the plurality of primary blades may be aligned with a corresponding cutting edge of each secondary blade of the plurality of secondary blades, for example.

In another aspect, the disclosure provides that in various embodiments, a first end of each link may be pivotally coupled to a first end of each blade, a second end of each link may be pivotally coupled to the collar, and a second end of each blade may be pivotally coupled to the positioning ring, for example.

In another aspect, the disclosure provides that in various embodiments the plurality of secondary blades are moveable between the retracted position and the extended position to an intermediate position, and in the intermediate position, a cutting edge of the plurality of secondary blades may be inclined with respect to a cutting edge of the plurality of primary blades, for example.

In another aspect, the disclosure provides that the reaming device may further include an adjusting ring, and the adjusting ring may be disposed around the cylindrical body and axially aligned with the longitudinal axis, for example.

In another aspect, the disclosure provides that an outer surface of the cylindrical body may be a first threaded surface and an interior surface of the adjusting ring may be a second threaded surface. In various embodiments, by rotating the adjusting ring with respect to the longitudinal axis in a first direction the adjusting ring urges the collar towards the distal end, and by rotating the adjusting ring with respect to the longitudinal axis in a second direction opposite the first direction the adjusting ring urges the collar towards the proximal end, for example.

In another aspect, the disclosure provides a method for reaming a boney surface. The method may include providing a variable diameter reaming device. The variable diameter reaming device may include a cylindrical body defining a longitudinal axis extending from a proximal end to a distal end, and a positioning ring disposed at the distal end of the cylindrical body that defines a first diameter, for example. The device may further include a plurality of primary blades radially disposed on the positioning ring, and the plurality of primary blades may be configured for reaming at the first diameter, for example. The device may further include a collar disposed around the cylindrical body that is axially aligned with the longitudinal axis, for example. The device may further include a plurality of secondary blades, and each secondary blade may be supported by the positioning ring and a corresponding link, for example. Additionally, each link may be operably connected to the collar, for example. The method may further include the step of moving the collar down the cylindrical body along the longitudinal axis towards a distal end, and moving the plurality of secondary blades from a retracted position to an extended position. In various embodiments, in the retracted position, the plurality of primary blades may be configured for reaming at the first diameter and the plurality of secondary blades are retracted, for example. In various embodiments, in the extended position, the plurality of primary blades may be configured for reaming at the first diameter and the plurality of secondary blades may be configured for reaming at a second diameter, for example. Additionally, the second diameter may be larger than the first diameter.

In another aspect, the disclosure provides for placing a broach stem within a centrally disposed aperture of the positioning ring and thereby positioning the plurality of primary blades radially around the broach stem, for example.

In another aspect, the disclosure provides for inclining the plurality of secondary blades with respect to the longitudinal axis in an intermediate position. In various embodiments, the intermediate position defines a third diameter that may be greater than the first diameter and less than the second diameter, for example.

In another aspect, the disclosure provides that moving the collar step may further include rotating an adjusting ring with respect to the longitudinal axis thereby urging the collar towards the distal end, for example.

The foregoing and other objects, features and advantages of the invention will be apparent from the following more particular descriptions of exemplary embodiments of the invention as illustrated in the accompanying drawings wherein like reference numbers generally represent like parts of the disclosure.

### BRIEF DESCRIPTION OF DRAWINGS

The following drawings are illustrative of particular embodiments of the present disclosure and therefore do not limit the scope of the present disclosure. The drawings are not to scale and are intended for use in conjunction with the explanations in the following detailed description.

FIG. 1 is an example side view of a femur and a calcar plane.

FIG. 2A is an example side view of a femur and a broach.

FIG. 2B is an example side view of the femur and broach of FIG. 2A after being reamed.

FIG. 3 is an example side perspective view of a first embodiment of a calcar planer.

FIG. 4 is an example side perspective view of a first embodiment of a calcar planer.

FIG. 5 is an example bottom perspective view of a first embodiment of a calcar planer.

FIG. 6 is an example top perspective view of a first embodiment of a calcar planer.

FIG. 7 is an example side view of a femur being reamed by the first embodiment of a calcar planer in an initial position.

FIG. 8 is an example side view of a femur being reamed by the first embodiment of a calcar planer in a partly expanded position.

FIG. 9 is an example side view of a femur being reamed by the first embodiment of a calcar planer in a fully expanded position.

FIG. 10 is an example side perspective view of a second embodiment of a calcar planer.

FIG. 11 is an example exploded parts view of a second embodiment of a calcar planer.

FIG. 12 is an example exploded parts view of a second embodiment of a calcar planer.

FIG. 13 is an example side view of a femur being reamed by the second embodiment of a calcar planer in an initial position.

FIG. 14 is an example side view of a femur being reamed by the second embodiment of a calcar planer in a partly expanded position.

FIG. 15 is an example side view of a femur being reamed by the second embodiment of a calcar planer in a fully expanded position.

### DETAILED DESCRIPTION

The following discussion omits or only briefly describes certain conventional features related to surgical systems for treating the spine, which are apparent to those skilled in the art. It is noted that various embodiments are described in detail with reference to the drawings, in which like reference numerals represent like parts and assemblies throughout the several views. Reference to various embodiments does not limit the scope of the claims appended hereto. Additionally, any examples set forth in this specification are intended to be non-limiting and merely set forth some of the many possible embodiments for the appended claims. Further, particular features described herein can be used in combination with other described features in each of the various possible combinations and permutations.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

Embodiments of the present disclosure relate generally, for example, to medical devices and methods for treating musculoskeletal disorders, and more particularly, to surgical systems and methods for treating the spine. Embodiments of the devices, methods, and systems are described below with reference to the Figures.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

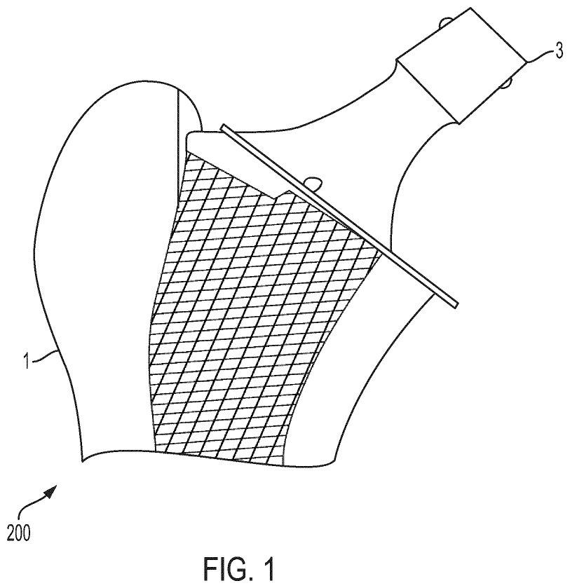

FIG. 1 is an example side view of a femur 1 including a broach 4 installed within a femoral canal of the femur 1. In the example illustration, the broach 4 may include a broach stem 3 extending out of the femoral canal, for example. As illustrated, femur 1 has been prepared such that the shoulder face of the broach 4 is aligned in substantially the same orientation and level as the calcar plane C-P. As used in this disclosure, “calcar” refers to the dense, vertically oriented bone present in the posteromedial region of the femoral shaft inferior to the lesser trochanter of the femur. Similarly, the “calcar plane” refers to the plane established after the performance of a femoral neck resection at the calcar region.

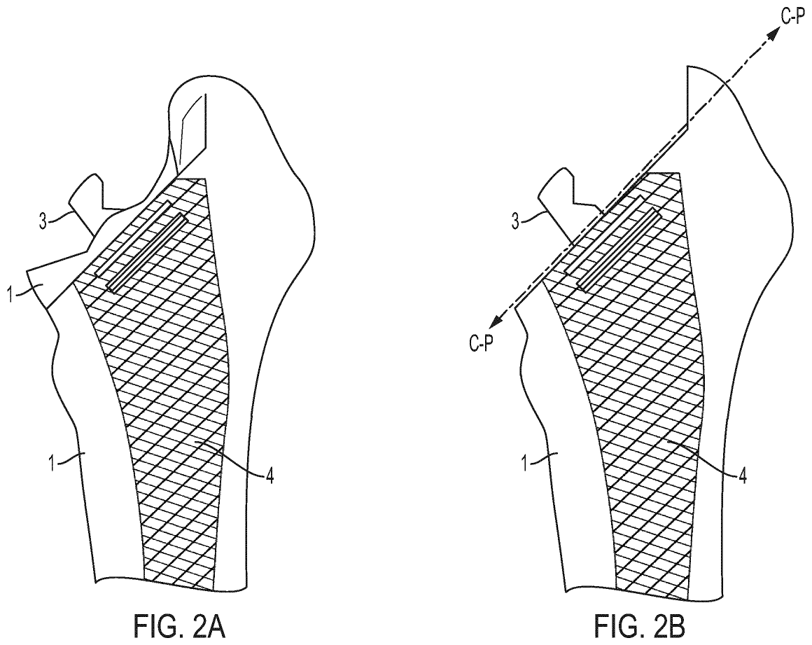

FIG. 2A is an example side view of a femur 1 and a broach 4. As illustrated, a femoral neck resection has been performed to femur 1. For example, a surgeon may have used saw blades to manually establish a general working area or surface without the use of a resection guide. Additionally, the general working area or surface may be a rough surface due to this common technique where the shoulder of the broach 4 is not aligned with a smooth plane of the femur. For example, a calcar region 2 includes a rough surface that is not aligned in a plane with the shoulder of the broach 4. In principle, to perform a successful THA it is generally important for the broach stem 3 to be seated at the proper elevation to avoid problems associated with leg length variation and interrupted range of motion. For example, the broach stem 3 should be seated to far above the head of the femur 1 or too deep below the head of the femur 1. FIG. 2B is an example side view of the femur 1 and broach 4 of FIG. 2A after the calcar region 2 has been reamed, for example. This reaming process, as will be explained in further detail below, assists a surgeon with ensuring that the calcar region 2 has been smoothed to a plane and that the broach stem 3 may be seated at the proper elevation where it is not too far above the head of the femur 1 or too deep below the head of the femur 1.

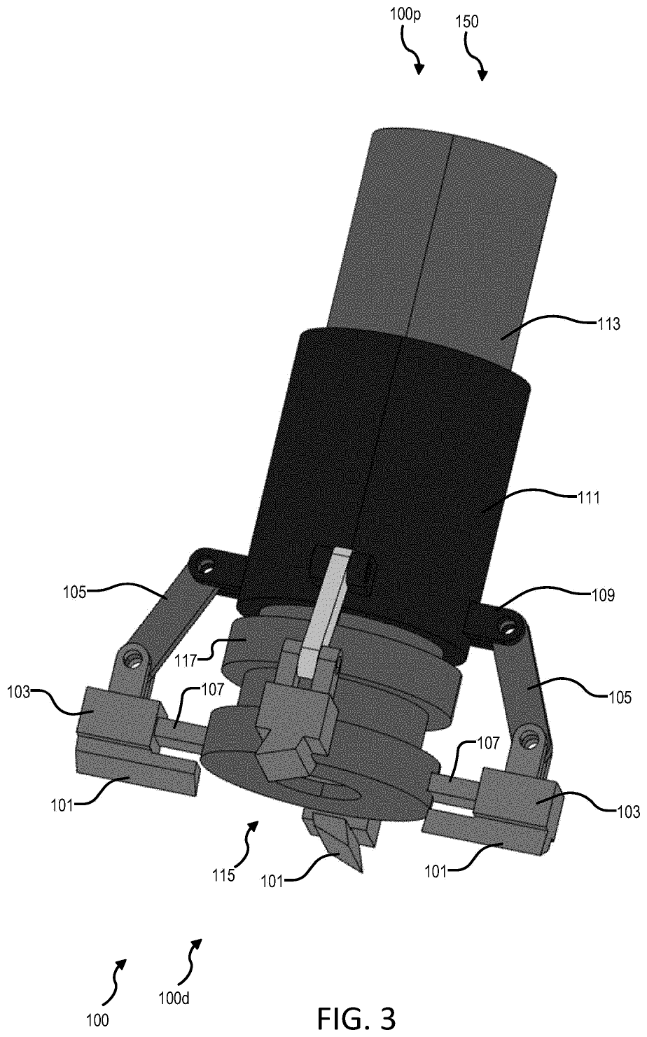

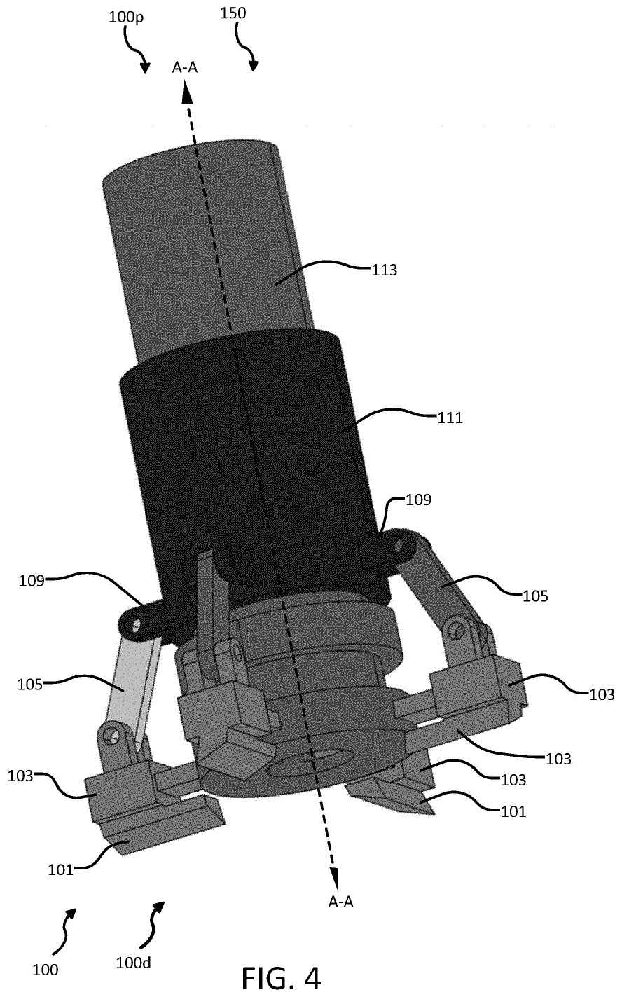

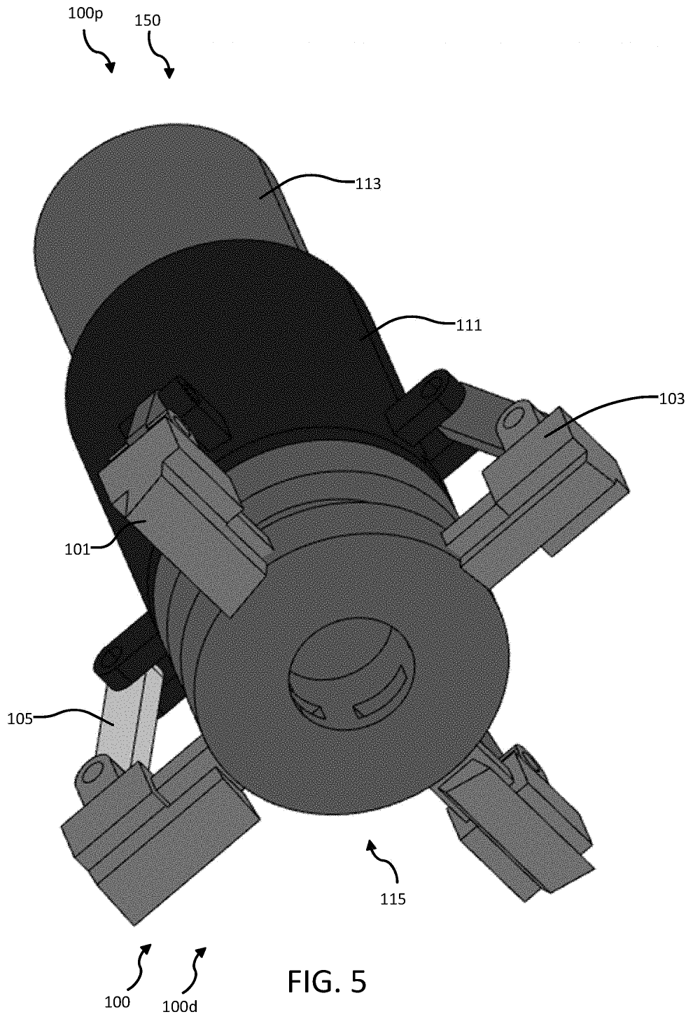

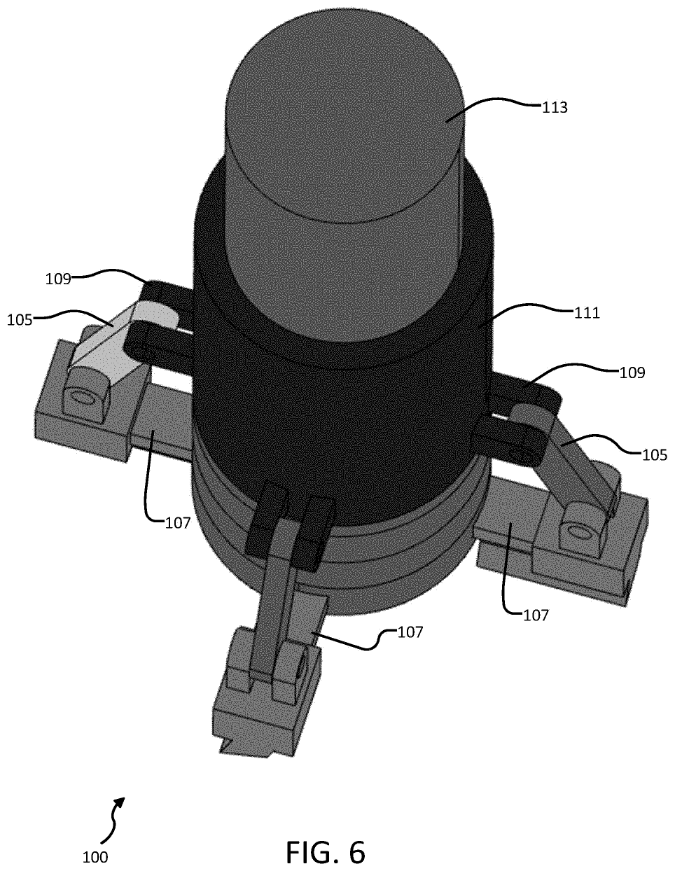

Referring generally to FIGS. 3-9 a first embodiment of a calcar planer 100 is illustrated. In the example embodiment, the calcar planer 100 has a proximal end 100p and a distal end 100d. The distal end 100d may include and/or be defined by a plurality of blades 101 configured for reaming a boney surface, for example. The calcar planer 100 may include a body 113 taking the shape of a cylinder extending in a longitudinal direction and defining a longitudinal axis A-A, for example. The body 113 may include a positioning ring 119 disposed at the distal end 100d, for example. The positioning ring 119 may include a centrally disposed aperture 115 that is axially aligned with the longitudinal axis 10. The centrally disposed aperture 115 may be configured for receiving and/or accepting a broach stem 3, for example. The positioning ring 119 may be operably coupled to a plurality of sliding arms 107 that can extend and retract within the positioning ring 119 in a direction that is substantially perpendicular to the longitudinal axis 10, for example.

In the example embodiment, each sliding arm 107 is operably coupled to a platform 103. Each platform 103 may support a cutting blade 101, for example. Each cutting edge of the cutting blades 101 may define a distal most end of the distal end 100d. Each platform 103 may be coupled to a corresponding link 105 and to a corresponding sliding arm 107. Each link may be operably coupled to a collar 111 at a corresponding collar arm 109, for example. In the example embodiment, each collar arm 109 extends laterally from a side surface of the collar 111. In other embodiments, the collar arms 109 may extend at an alternate inclined angle or may be omitted altogether. In the example embodiment, the collar 111 may be disposed around the cylindrical body and axially aligned with the longitudinal axis, for example. The collar 111 may slide forward and backward along the outside of the body 113 and thereby extend the blades 101 laterally outward and laterally inward, as will be explained in further detail below.

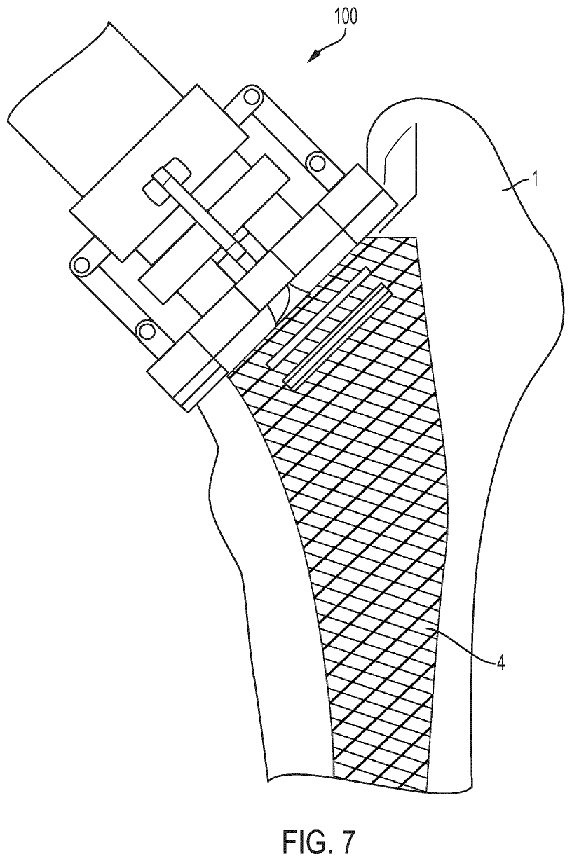

FIG. 7 illustrates the variable diameter calcar planer 100 in a retracted position (first position). As illustrated, the variable diameter calcar planer 100 is positioned around a broach stem 3. For example, the broach stem 3 is positioned inside of the aperture 115. In the retracted position, the collar 111 is slid, moved, or positioned up the body 113 towards the proximal end 100p. For example, the links 105 extend in a direction that is substantially parallel with the longitudinal axis A-A and the collar 111 is as far towards the proximal end 100p as the links 105 permit. In the retracted position, the plurality of blades 101 are configured for reaming a boney surface at a first diameter.

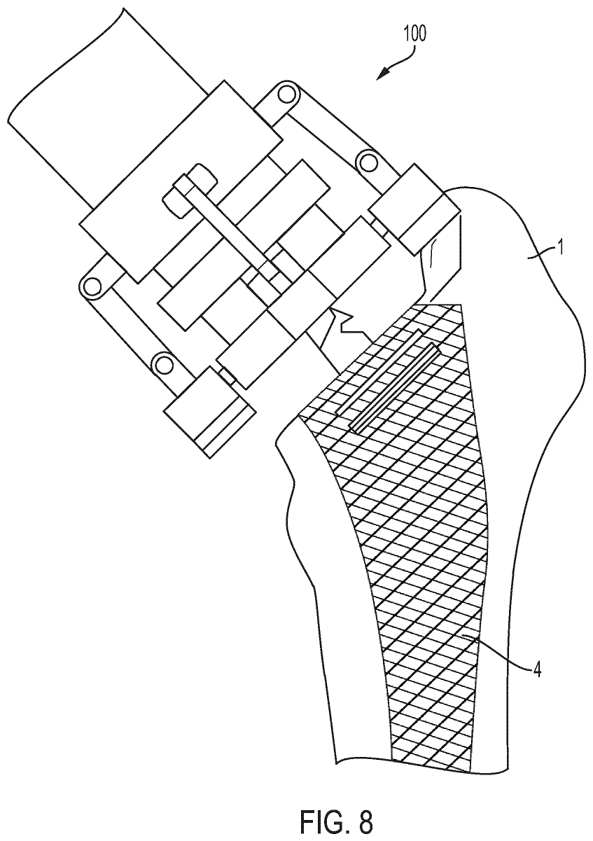

FIG. 8 illustrates the variable diameter calcar planer 100 in an intermediate position (second position). As illustrated, the collar 111 is slid, moved, or positioned down the body 113 away from the proximal end 100p and towards the distal end 100d. By sliding the collar 111 towards the distal end 100d, the sliding arms 107 begin to extend outward laterally in a direction that is substantially perpendicular from the longitudinal axis A-A. For example, each sliding arm 107 is configured to support and move each platform 103 towards and away from the positioning ring 119 in a lateral direction that is substantially perpendicular with the longitudinal axis A-A. Additionally, the links 105 move from the retracted position to an inclined position. For example, the pinned connections of the links 105 at the platform 103 and collar arm 109 facilitate the articulation of the links 105 outward. In the intermediate position, the plurality of blades 101 are configured for reaming a boney surface at a second diameter that is greater than the first diameter.

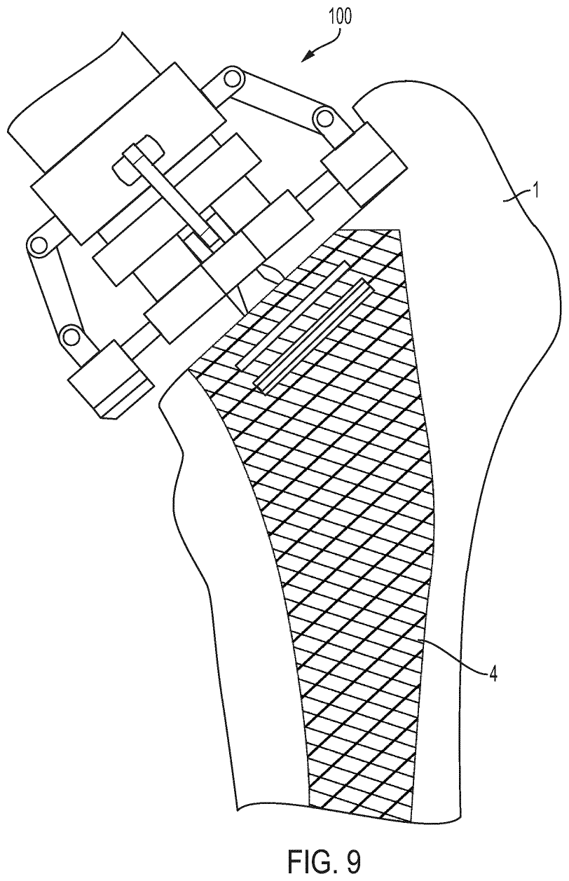

FIG. 9 illustrates the variable diameter calcar planer 100 in an extended position (third position). As illustrated, the collar 111 may slide, move, or otherwise be positioned down the body 113 away from the proximal end 100p and towards the distal end 100d. In some embodiments, the stop ring 117 may be positioned along body 113 to control the position of collar 111 and therefore the diameter of a circular reaming area defined by the plurality of blades 101. For example, the stop ring 117 may prevent the collar 111 from moving to far in the distal direction 100d. Consistent with the previous disclosure, by sliding the collar 111 towards the distal end 100d, the sliding arms 107 continue to extend outward laterally in a direction that is substantially perpendicular to the longitudinal axis A-A. Additionally, the links 105 move from the intermediate position to an extended position. For example, the pinned connections of the links 105 at the platform 103 and collar arm 109 facilitate the articulation of the links 105 outward. In the extended position, the plurality of blades 101 are configured for reaming a boney surface at a third diameter that is greater than the first diameter.

In practice, an end user such as a surgeon may position the variable diameter calcar planer 100 to surround a broach stem 3. Next, the surgeon may rotate the blades 101 by coupling the variable diameter calcar planer 100 to an external driver (not illustrated). An example external driver may be a powered driver or a hand driver, for example. At least one embodiment couples a drive end 150 to a powered instrument such as the POWEREASE™ System sold by Medtronic and/or the powered rotary-type handpiece described in U.S. Pat. No. 10,456,122, for example. In coupling the variable diameter calcar planer 100 to the drive instrument, a surgeon may cause the blades 101 to rotate and begin a reaming process at a first diameter, for example in the retracted position shown in FIG. 7. Next, a surgeon may slide the collar 111 down as previously explained and activate the drive instrument to cause rotation of the blades 101 and continue reaming the boney surface. Thereafter, a surgeon may continue to slide the collar 111 down in the distal direction 100d until the blades 101 are extended laterally far enough outward to ream the boney surface until a calcar plane C-P is established (see FIG. 1).

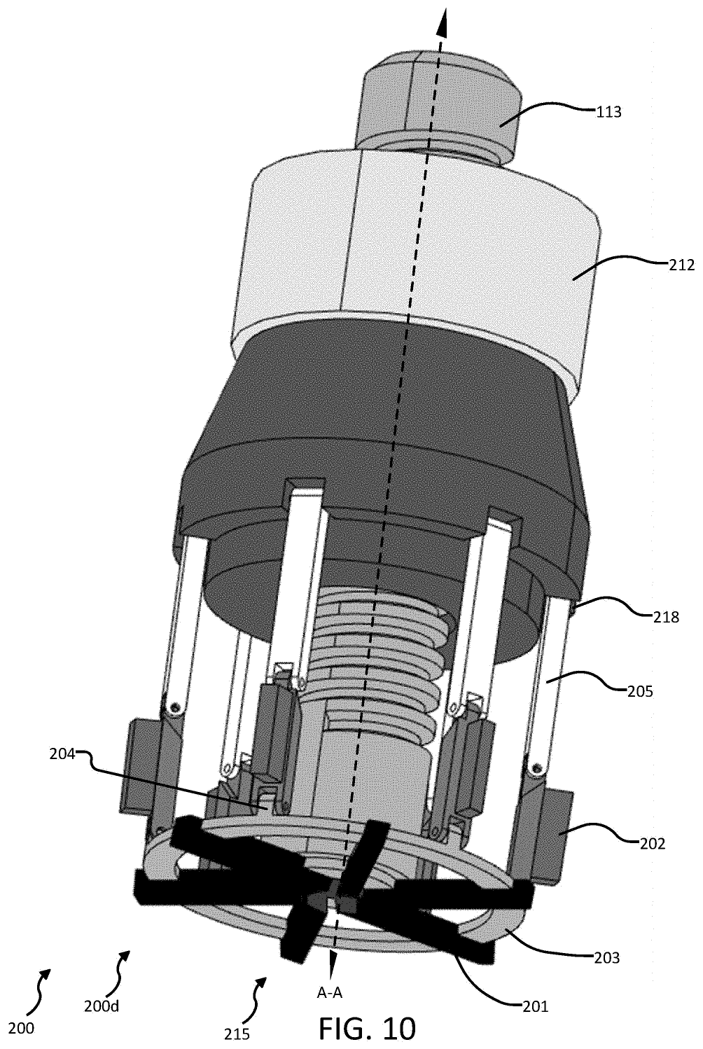

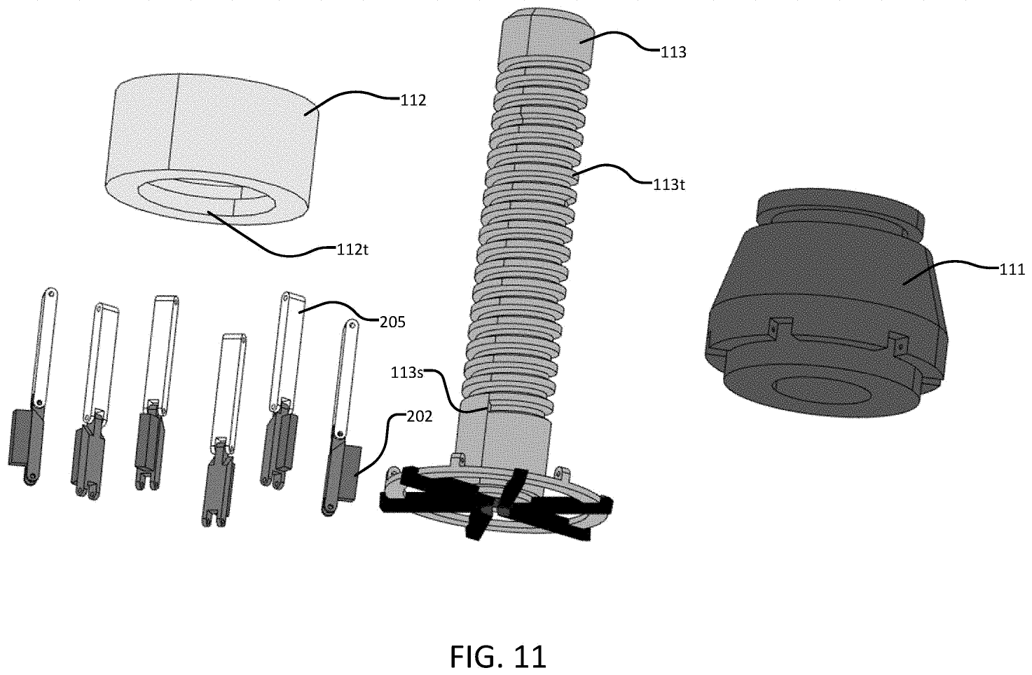

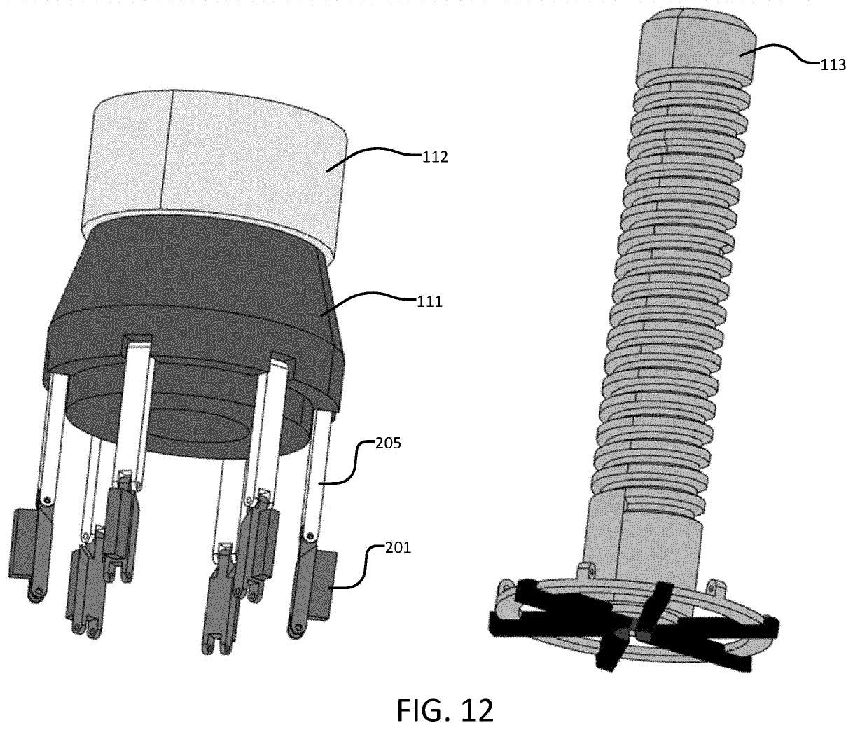

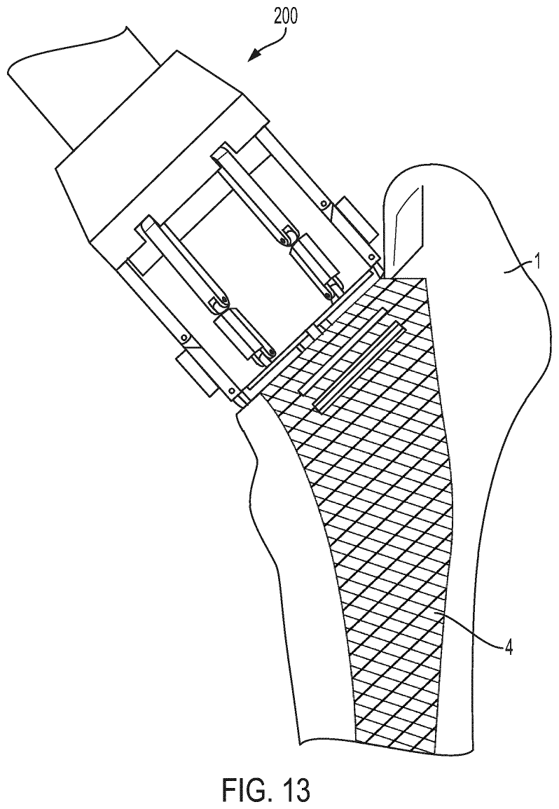

Referring generally to FIGS. 10-15 a second embodiment of a variable diameter calcar planer 200 is disclosed. FIG. 10 is an example side perspective view of a second embodiment of a calcar planer 200. FIGS. 11 and 12 are example exploded parts view of a second embodiment of a calcar planer 200. FIGS. 13-15 are example illustrates of the calcar planer 200 in operation. This second embodiment may include the same, substantially the same, and/or similar components and functionality as explained above with respect to the first embodiment. Accordingly, their attributes are equally applicable to both example embodiments unless the context and functionality clearly indicates otherwise.

In the example embodiment, the calcar planer 200 has a proximal end 200p and a distal end 200d. The distal end 200d may include and/or be defined by a plurality of primary blades 201 that are configured for reaming a boney surface, for example. The calcar planer 200 may include a body 113 taking the shape of a cylinder extending in a longitudinal direction and defining a longitudinal axis A-A, for example. In the example embodiment, the outside lateral surface of the body 113 comprises a threaded surface extending down the majority of the body 113, for example. A first end of body 113 may include a drive end 250 for operably connecting the calcar planer 200 to a drive tool at a proximal end 200p of the calcar planer 200 similarly as explained above with respect to calcar planer 100. Additionally body 113 may include an aperture 215 for operably positioning the calcar planer 200, for example around a broach stem 3, similarly as explained above with respect to calcar planer 100. Similarly, the body 113 may include a positioning ring 203 disposed at the distal end 200d, for example. The positioning ring 203 may include a centrally disposed aperture that is axially aligned with longitudinal axis A-A and aperture 215 of body 113, for example.

Calcar planer 200 may include a plurality of radially disposed primary blades 201 that are symmetrically distributed around positioning ring 203. For example, each of primary blades 201 may extend from a central region corresponding to aperture 215 to an edge portion of positioning ring 203. In the example embodiment, six blades 201 are illustrated but it is contemplated that any number of blades 201 may be disposed on positioning ring 203. Additionally, the primary blades 201 need not be symmetrically distributed.

In the example embodiment, positioning ring 203 may include a plurality of connection points 204 on a top surface thereof (a surface facing the proximal direction 200p, for example. The connection points 204 may be a pivotable connection such as a pin and socket connection, a hinge, or the like. Each connection point 204 may be operably coupled to a corresponding secondary blade 202 (also referred to as a pivoting blade) at a first end (distal end). In turn, each secondary blade 202 may be operably coupled to a corresponding link 205 by a pivotable connection at a second end (proximal end) of the secondary blade 202. Additionally, each link 205 may be coupled to collar 111 at a pivotable connection, for example. In the example embodiment, each link 205 is coupled to collar 111 within a nested cavity 218. At least one advantage of coupling links 205 to collar 111 within a nested cavity is a reduction and/or elimination from lateral protrusions of the collar 111 as safety feature. For example, a lateral protrusion could snag a surgeons hand while the calcar planer 200 is in operation and rotating. Additionally, nested cavity 218 may prevent the links 205 from extending too far inward towards longitudinal axis A-A, for example.

In the example embodiment, an adjusting ring 112 may be provided for moving collar 111 forward and backward along the longitudinal axis A-A, for example. In the example embodiment, adjusting ring 112 may include an interior threaded surface 112t that corresponds in size, shape, and orientation to the threaded surface of body portion 113 (see FIG. 11). Additionally, adjusting ring 112 may be coupled to collar 111. In some embodiments, collar 111 may not include a threaded interior surface and in other embodiments collar 111 may include an interior threaded surface the same as or similar to interior threaded surface 112t of adjusting ring 112. In the example embodiment, the adjusting ring 112 is rotatable around body 113 to move collar 111 forward and backward along body 113 in a longitudinal direction. For example, due to the corresponding threaded surfaces of body 113 and adjusting ring 112 by rotating adjusting ring 112 the collar 111 will slide and/or move in a longitudinal direction. Similarly as explained above with respect to calcar planer 100, by moving the collar 111 the secondary blades 202 will pivot outward with respect to longitudinal axis A-A to thereby increase an area for reaming by calcar planer 200.

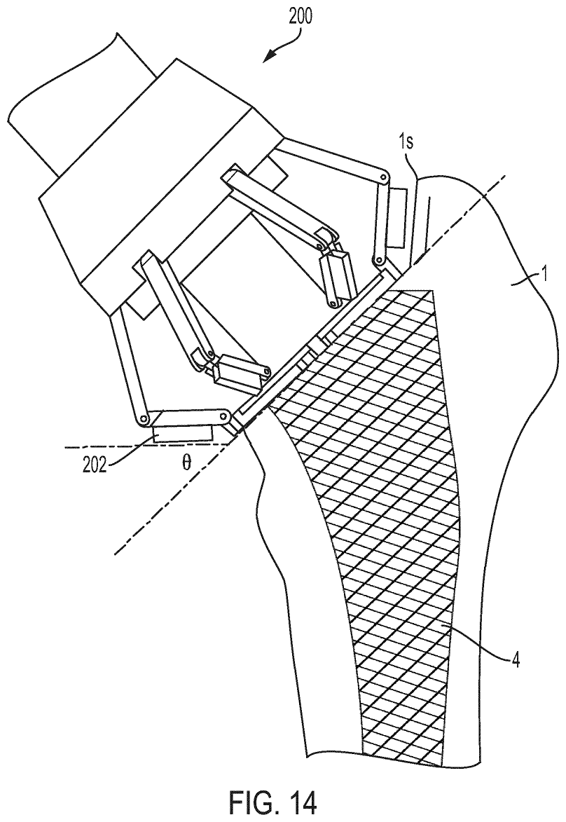

Those with skill in the art will appreciate that each corresponding link 205 and secondary blade 202 combination comprises three pivotable connections. The three pivotable connections increase the range of motion of secondary blades 202. A distal connection between secondary blades 202 and positioning ring 203 is fixed and/or supported in terms of spatial location but allows pivoting outward and away (and inward and towards) from longitudinal axis A-A. The distal connection may include a stop feature that prevents the pivoting of link 205 too far inward towards longitudinal axis A-A. A proximal connection between links 205 and collar 111 is fixed and/or supported in terms of spatial location but allows pivoting outward and away (and inward and towards) from longitudinal axis A-A. Similarly, the proximal connection may include a stop feature that prevents the pivoting of link 205 too far inward towards longitudinal axis A-A. A central connection between secondary blades 202 and link 205 (proximal side of secondary blade 202 and distal side of link 205) may not be fixed to a particular structural support and therefore allow pivoting outward and away (and inward and towards) from longitudinal axis A-A due to the distal connection and proximal connection being fixed and/or supported in terms of spatial location. These three pivotable connections allow secondary blades 202 to extend outward with respect to longitudinal axis A-A and to pivot, articulate, or otherwise be inclined with respect to longitudinal axis A-A. Similarly, these three pivotable connections allow secondary blades 202 to extend outward with respect to longitudinal axis A-A within a range of various angles θ depending on the position of collar 111. For example, the range of various angles θ be measured with respect a first reaming plane defined by the primary blades 201. Additionally, the range of various angles θ may be between about 90 degrees and about 0 degrees with respect to the first reaming plane. In this way, the secondary blades 202 may form an angled reaming area or angled reaming ring (See FIG. 14) with respect to the first reaming plane. Another way to visualize and/or explain the reaming area corresponding to secondary blades 202 in the angled position is with respect to an imaginary cone. For example, secondary blades 202 may ream an area corresponding to a side surface of an imaginary cone that is inverted with respect to calcar planer 200. For example, a plane of the base of the cone may be flush with an end surface of calcar planer 200 at the proximal end 200p and be centered with respect to longitudinal axis A-A. Side surfaces of the cone may extend from the base to a tip portion of the imaginary cone and the tip portion may extend past the proximal end 200p and be axially aligned with longitudinal axis A-A. In this way, the angled reaming surface corresponding to the secondary blades 202 may correspond to the side surface of an imaginary cone.

For example, when secondary blades 202 are angled as illustrated in FIG. 14 the cutting edge of secondary blades 202 may perform reaming of a side surface of a boney structure while primary blades 201 perform reaming of a horizontal surface of the boney structure in a first reaming plane. As illustrated in FIG. 14, this configuration may be particularly advantageous for cutting the side surface 1s of femur 1. In some embodiments, the side surface 1s may also correspond to a knuckle portion of femur 1. Another way to visualize and/or explain these two reaming areas is the primary blades 201 are configured for reaming a boney surface in a first plane that is substantially perpendicular to the longitudinal axis A-A and the secondary blades 202 are adjustable for reaming with respect to the first plane. For example, a cutting surface of secondary blades 202 is inclinable between 0 degrees and 90 degrees with respect to a cutting surface of primary blades 201.

FIG. 14 illustrates the calcar planer 200 in a retracted position (first position). As illustrated, the calcar planer 200 is positioned around a broach stem 3. For example, the broach stem 3 is positioned inside of the aperture 115. In the retracted position, the collar 111 is rotated, moved, or positioned up the body 113 towards the proximal end 100p such that the secondary blades 202 are fully retracted, i.e., a cutting surface of the secondary blades extends in a direction that is parallel to the longitudinal axis. For example still, the links 205 extend in a direction that is substantially parallel with the longitudinal axis A-A and the collar 111 is as far towards the proximal end 100p as the links 205 permit. It should be understood that in the retracted position, the plurality of primary blades 201 are configured for reaming a boney surface in a first plane that is substantially perpendicular to the longitudinal axis at a first diameter.

FIG. 14 illustrates the calcar planer 200 in an intermediate position (second position) where the secondary blades 202 are inclined with respect to the primary blades 201. As illustrated, the collar 111 is rotated, moved, or positioned down the body 113 away from the proximal end 100p and towards the distal end 100d. By moving the collar 111 towards the distal end 100d, the secondary blades 202 begin to move laterally outward with respect to longitudinal axis A-A due to the various pinned connections as explained above. For example, the pinned connections of the links 205 at the collar 111 and positioning ring 203 facilitate the articulation of the links 205 outward. In the intermediate position, the plurality of primary blades 201 are configured for reaming a boney surface at the first diameter in the first plane and the plurality of secondary blades 202 are configured for reaming in a direction that is angled with respect to the first plane.

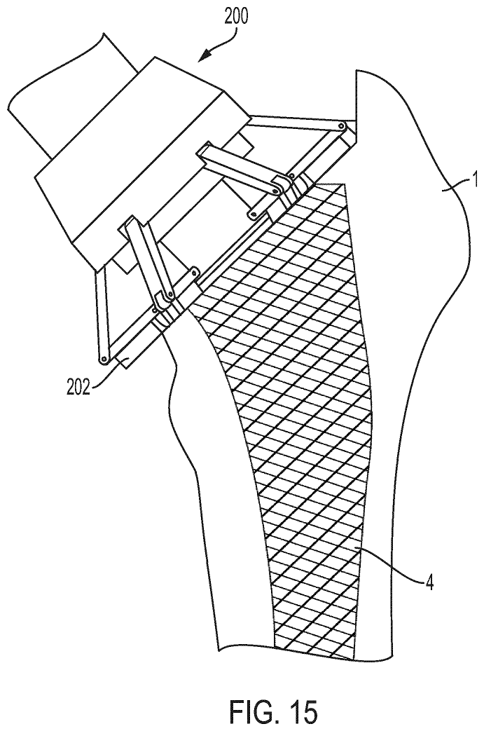

FIG. 15 illustrates the calcar planer 200 in an extended position (third position). As illustrated, the collar 111 may rotate, move, or otherwise be positioned down the body 113 away from the proximal end 100p and towards the distal end 100d. In some embodiments, the threaded portion of the body 113 may include a stop feature 113s preventing the collar 111 from moving to far down the body 113, for example. Consistent with the previous disclosure, by moving the collar 111 towards the distal end 100d, the secondary blades 202 continue to extend outward laterally until a cutting surface of the secondary blades 202 is oriented in a parallel direction with a cutting surface of the primary blades 201. In the extended position, the plurality of primary blades 201 are configured for reaming a boney surface at the first diameter and in a first plane. Additionally, the plurality of secondary blades 202 are configured for reaming a boney surface at a second diameter and in a first plane. In the extended position, the primary and secondary blades collectively may ream a boney surface at a second diameter and in the first plane that is greater than the first diameter.

In practice, an end user such as a surgeon may position the variable diameter calcar planer 200 to surround a broach stem 3. Next, the surgeon may rotate the body 113 by coupling a drive end 250 of the variable diameter calcar planer 200 to an external driver (not illustrated). An example external driver may be a powered driver or a hand driver, for example. At least one embodiment couples drive end 250 to a powered instrument such as the POWEREASE™ System sold by Medtronic and/or the powered rotary-type handpiece described in U.S. Pat. No. 10,456,122, for example. In coupling the variable diameter calcar planer 200 to the drive instrument, a surgeon may cause the blades 201 and 202 to rotate and begin a reaming process at a first diameter, for example in the retracted position shown in FIG. 13. Next, a surgeon may move the collar 111 down as previously explained and activate the drive instrument to cause rotation of the blades 201 and 202 and continue reaming the boney surface in the first plane and also reaming a side surface of the boney structure (see FIG. 14). Thereafter, a surgeon may continue to move the collar 111 down in the distal direction 200d until the secondary blades 202 are extended laterally outward until a cutting surface of the secondary blades 202 is aligned with a cutting surface of the primary blades 201. In the extended position, the primary and secondary blades 201 and 202 collectively form an enlarged reaming area compared to the reaming area defined solely by the primary blades 201.

What is claimed is:

1. A variable diameter reaming device, comprising:
a cylindrical body defining a longitudinal axis extending from a proximal end to a distal end;
a positioning ring disposed at the distal end of the cylindrical body, the positioning ring having a centrally disposed guide aperture;
a collar disposed around the cylindrical body and axially aligned with the longitudinal axis; and
a plurality of radially disposed blades, each blade being supported by a corresponding platform, each platform being operably connected to the positioning ring by a sliding arm and being operably connected to the collar by a link;
wherein the collar is configured to move up and down the cylindrical body along the longitudinal axis thereby moving the plurality of blades between a retracted position and an extended position,
wherein, in the retracted position, the plurality of blades are configured for reaming at a first diameter, and
wherein, in the extended position, the plurality of blades are configured for reaming at a second diameter, the second diameter being larger than the first diameter.

2. The variable diameter reaming device of claim 1, wherein the cylindrical body further includes a stop ring disposed adjacent to the positioning ring, the stop ring being configured to stop the cylindrical collar in the extended position.

3. The variable diameter reaming device of claim 1, wherein the cylindrical body further includes a plurality of collar arms, each collar arm being coupled to a corresponding link by a pinned connection.

4. The variable diameter reaming device of claim 3, wherein each platform is operably coupled to a corresponding link by a pinned connection.

5. The variable diameter reaming device of claim 4, wherein each sliding arm is configured to support and move each platform towards and away from the positioning ring in a lateral direction, the lateral direction being substantially perpendicular with the longitudinal direction.

6. The variable diameter reaming device of claim 5, wherein the guide aperture is configured to surround a broach stem thereby positioning the plurality of blades radially around the broach stem.

7. A variable diameter reaming device, comprising:
a cylindrical body defining a longitudinal axis extending from a proximal end to a distal end;
a positioning ring disposed at the distal end of the cylindrical body, the positioning ring defining a first diameter;
a plurality of primary blades radially disposed on the positioning ring, the plurality of primary blades being configured for reaming at the first diameter;
a collar disposed around the cylindrical body and axially aligned with the longitudinal axis; and
a plurality of secondary blades, each secondary blade being supported by the positioning ring and a corresponding link, each link being operably connected to the collar,
wherein the collar is configured to move up and down the cylindrical body along the longitudinal axis thereby moving the plurality of secondary blades between a retracted position and an extended position,
wherein, in the retracted position, the plurality of primary blades are configured for reaming at the first diameter and the plurality of secondary blades are retracted,
wherein, in the extended position, the plurality of primary blades are configured for reaming at the first diameter and the plurality of secondary blades are configured for reaming at a second diameter, the second diameter being larger than the first diameter.

8. The variable diameter reaming device of claim 7, wherein the positioning ring comprises a centrally disposed guide rail and an outer rail, the centrally disposed guide rail defining a centrally disposed aperture configured to surround a broach stem thereby positioning the plurality of primary blades and the plurality of secondary blades radially around the broach stem.

9. The variable diameter reaming device of claim 8, wherein each primary blade of the plurality of primary blades extends from the centrally disposed guide rail to the outer rail.

10. The variable diameter reaming device of claim 7, wherein a cutting edge of each primary blade of the plurality of primary blades extends in a direction that is substantially perpendicular to the longitudinal axis.

11. The variable diameter reaming device of claim 10, wherein in the extended position, a cutting edge of each secondary blade of the plurality of secondary blades extends in a direction substantially perpendicular to the longitudinal axis.

12. The variable diameter reaming device of claim 11, wherein in the extended position each cutting edge of each primary blade of the plurality of primary blades is aligned with a corresponding cutting edge of each secondary blade of the plurality of secondary blades.

13. The variable diameter reaming device of claim 7, wherein:
a first end of each link is pivotally coupled to a first end of each blade;
a second end of each link is pivotally coupled to the collar; and
a second end of each blade is pivotally coupled to the positioning ring.

14. The variable diameter reaming device of claim 13, wherein:
the plurality of secondary blades are moveable between the retracted position and the extended position to an intermediate position; and
in the intermediate position, a cutting edge of the plurality of secondary blades is inclined with respect to a cutting edge of the plurality of primary blades.

15. The variable diameter reaming device of claim 7, further comprising an adjusting ring, the adjusting ring being disposed around the cylindrical body and being axially aligned with the longitudinal axis.

16. The variable diameter reaming device of claim 7, wherein:
an outer surface of the cylindrical body comprises a first threaded surface;
an interior surface of the adjusting ring comprises a second threaded surface;
by rotating the adjusting ring with respect to the longitudinal axis in a first direction the adjusting ring urges the collar towards the distal end; and
by rotating the adjusting ring with respect to the longitudinal axis in a second direction opposite the first direction the adjusting ring urges the collar towards the proximal end.

17. A method for reaming a boney surface, comprising:
providing a variable diameter reaming device, the variable diameter reaming device comprising:
a cylindrical body defining a longitudinal axis extending from a proximal end to a distal end,
a positioning ring disposed at the distal end of the cylindrical body, the positioning ring defining a first diameter;
a plurality of primary blades radially disposed on the positioning ring, the plurality of primary blades being configured for reaming at the first diameter;
a collar disposed around the cylindrical body and axially aligned with the longitudinal axis;
a plurality of secondary blades, each secondary blade being supported by the positioning ring and a corresponding link, each link being operably connected to the collar;
moving the collar down the cylindrical body along the longitudinal axis towards a distal end; and
moving the plurality of secondary blades from a retracted position to an extended position,
wherein, in the retracted position, the plurality of primary blades are configured for reaming at the first diameter and the plurality of secondary blades are retracted, and
wherein, in the extended position, the plurality of primary blades are configured for reaming at the first diameter and the plurality of secondary blades are configured for reaming at a second diameter, the second diameter being larger than the first diameter.

18. The method of claim 17, further comprising:
placing a broach stem within a centrally disposed aperture of the positioning ring thereby positioning the plurality of primary blades radially around the broach stem.

19. The method of claim 18, further comprising:
inclining the plurality of secondary blades with respect to the longitudinal axis in an intermediate position, and
wherein the intermediate position defines a third diameter that is greater than the first diameter and less than the second diameter.

20. The method of claim 19, wherein the moving the collar step further comprises rotating an adjusting ring with respect to the longitudinal axis thereby urging the collar towards the distal end.
